(19) United States
(12) Patent Application Publication
(10) Pub. No.: US 2025/0053995 A1
(43) Pub. Date: Feb. 13, 2025

(54) ITEM COLLECTION, RETURN, EXCHANGE WITHOUT HUMAN INTERACTION

(71) Applicant: SYNQ ACCESS + SECURITY TECHNOLOGY LTD., Victoria (CA)

(72) Inventors: Nolan Richard WHEELER, Saanich (CA); Joshua Diego Alexander RODRIGUEZ, Victoria (CA)

(21) Appl. No.: 18/717,838

(22) PCT Filed: Dec. 8, 2022
(86) PCT No.: PCT/CA2022/051798

§ 371 Date: Jun. 7, 2024

Related U.S. Application Data
(60) Provisional application No. 63/287,681, filed on Dec. 9, 2021.

(51) Int. Cl. G06Q 30/016; A61L 2/10; A61L 2/24; B65G 1/04; E05F 15/76; G08B 21/18

(52) U.S. Cl. CPC G06Q 30/016; A61L 2/10; A61L 2/24; B65G 1/04; E05F 15/76; A61L 2202/11; A61L 2202/121; A61L 2202/122; A61L 2202/14; G08B 21/18

(57) ABSTRACT

The recent pandemic has resulted in a desire to avoid human-to-human contact where possible. Purchasing items from a brick-and-mortar store traditionally involves close human interaction and touching items that have been touched by others. Now, to reduce human-to-human interaction, a bank of compartments is described into which items are placed by staff members for later collection by customers. After agreeing a purchase, a staff member places the purchased item in a compartment. The customer uses a smartphone to announce their arrival at the bank of compartments and provide authentication, at which point the compartment with the item is identified to the customer. The compartment automatically unlocks and opens allowing the customer to obtain the item without touching the compartment or its door. Deposits of purchased items are made via the bank for exchange, repair or refund. Each compartment may sanitize itself and its contents with UV-C light.

### TECHNICAL FIELD

The field of the invention is the collection, return and exchange of items. In particular, it relates to the collection, return and exchange of items without direct human-to-human interaction.

### BACKGROUND

COVID-19 has caused a shift in focus in people when deciding to venture into public, to interact with other people, or to even touch certain things. Limiting the interaction with other people has taken on a new importance. For example, it is now possible to order coffee or a fast-food take-out meal on one's phone. However, one still must interact with staff to pick up a coffee or take-out meal.

U.S. Pat. No. 10,719,804 to Lundahl describes a smartphone-controlled mailbox system that includes a remote computing device and a mailbox. The remote computing device is configured to communicate with a user device and to receive and review user authorization information and transmit an authorization message indicating successful authorization. The mailbox has a mail piece receiving mechanism, a mailbox identifier, a label printer, a locking mechanism, and a microprocessor. The microprocessor is configured to communicate with a user device having received an authorization message, to control the label printer to print a mailing label corresponding to services selected by a user via the user device, to control the locking mechanism in response to communications from the user device to allow the user to place a mail piece in the mail piece receiving mechanism and to lock the mail piece receiving mechanism after the mail piece is placed therein, and to communicate with the remote computing device.

U.S. Pat. No. 9,704,319 to Desinor describes a real estate wireless lockbox that can be used to manage real estate sales tours. When potential buyers approach the property, software on their computing devices can communicate via a wireless network and unlock the wireless lockbox.

US patent application publication No. 2021/0235891 to Derosa et al. describes a self-serve food locker. Embodiments are directed to a touchless automatic food locker apparatus providing automated self-serve food lockers. Embodiments include arrays of modular food lockers having a public access door and a kitchen access door opposed to each other. Heating and cooling elements maintain the appropriate food item temperature in the locker. Sensors, lighting, and ultraviolet lighting coupled to the locker detect a state of the item in the locker. The locker further includes credentialed access by the customer utilizing mobile communication and intelligent analysis to reduce waiting time, keep food products fresh, and assure quality product delivery.

US patent application publication No. 2020/0237119 to Jakubowski et al. describes a to-go cubby unit. A cubby unit includes a plurality of cubbies and a thermal management system. Each of the plurality of cubbies includes a cubby housing defining an internal compartment. The thermal management system is configured to facilitate thermally regulating at least one of (i) a food product selectively received within the internal compartment of at least one of the of cubbies or (ii) the internal compartment of at least one of the cubbies.

US patent application publication No. 2019/0236873 to Estill describes an item depository apparatus operated responsive to data bearing records. A depository operates to accept and make available deposit items to authorized users responsive at least in part to data read from data bearing records. At least one reading device is operative to read indicia included on depository items so that depository items placed in or removed from the interior area can be tracked.

US patent application publication No. 2012/0030130 to Smith et al. describes a system and method for identifying, reporting, and evaluating a presence of a solid, liquid, gas, or other substance of interest, particularly a dangerous, hazardous, or otherwise threatening chemical, biological, or radioactive substance. The system comprises one or more substantially automated, location self-aware remote sensing units, a control unit, and one or more data processing and storage servers.

This background is not intended, nor should be construed, to constitute prior art against the present invention.

### SUMMARY

There is provided a method for facilitating collection, return and exchange of items without human interaction. The method involves providing a bank of compartments, with the bank of compartments having a plurality of individual compartments. The method involves instructing the customer to use a personal electronic device upon arrival at the compartments to communicate with a server. The method involves programming the server to respond to the communication from the customer by providing a temporary communication interface. Upon the customer providing information deemed necessary to the server via the temporary communication interface, the server directs the customer to a selected one of the compartments.

The method, as described above, is suitable for use in situations in which theft is not a major concern. Where theft is a concern, each of the individual compartments has a door locked by an electronic lock controlled by the server. Upon the customer providing the information deemed necessary to the server via the temporary communication interface, the server releases the electronic lock for the door of the selected compartment.

Where a “touchless” experience is desired to avoid the need to sanitize the door after each use, a drive motor may be connected to the door of each of the individual compartments to move the door of the selected one of the plurality of individual compartments between a closed position and an open position.

The method allows customers to collect and return items at their own convenience. This may be at retail stores, storage units in workplaces, mailboxes in high-density residential environments, subways, bus stations, gyms, and high security environments where personal belongings are not allowed in.

Where a customer seeks to deposit an item for storage and later collection, the method involves a further step in which, after expiration of a predetermined time duration, the server is programmed to cause the door of the selected one of the plurality of individual compartments to be moved to a closed position by the drive motor and for the electronic lock to relock.

UV-C (ultraviolet-C) radiation is provided in some compartments to sanitize the compartment, the contents of the compartments, or both.

Disclosed is a method for facilitating a return by a customer of an item that has been purchased, comprising: establishing, by a server, a communication link with the customer; determining, by the server, that the customer is present at a bank of compartments; opening, by instruction from the server, a compartment of the bank of compartments; determining, by the server, that the item has been placed in the compartment; instructing, by the server, the compartment to close; informing, by the server, a staff member that the item has been returned; and identifying, by the server, the compartment to the staff member.

Also disclosed is a system for facilitating a return by a customer of an item that has been purchased, comprising a bank of compartments, and a server comprising a processor and computer-readable memory storing computer-readable instructions which, when executed by the processor cause the server to: establish a communication link with the customer; determine that the customer is present at the bank of compartments; open a compartment of the bank of compartments; determine that the item has been placed in the compartment; instruct the compartment to close; inform a staff member that the item has been returned; and identify the compartment to the staff member.

Further disclosed is a non-transitory, computer-readable medium storing computer-readable instructions, which when executed by a processer, cause a server to: establish a communication link with the customer; determine that the customer is present at a bank of compartments; open a compartment of the bank of compartments; determine that the item has been placed in the compartment; instruct the compartment to close; inform a staff member that the item has been returned; and identify the compartment to the staff member.

This summary provides a simplified, non-exhaustive introduction to some aspects of the invention, without delineating the scope of the invention.

### BRIEF DESCRIPTION OF THE DRAWINGS

The above and other features will become more apparent from the following description in which reference is made to the appended drawings, which are for the purpose of illustration only and are not intended to be in any way limiting, wherein:

FIG. 1 is a flow diagram outlining the sequence of a method for collecting an item, according to an embodiment of the present invention.

FIG. 2 is a schematic front elevation view of a bank of multiple storage units, according to an embodiment of the present invention.

FIG. 3 is a side elevation view, in section, of an interior of an individual compartment, according to an embodiment of the present invention.

FIG. 4 is a schematic diagram of a personal electronic device that may be used according to an embodiment of the present invention.

FIG. 5 is a front elevation view of the interior of an individual compartment with the door open, according to an embodiment of the present invention.

FIG. 6 is a block diagram showing the communication and control interface and connection to a personal electronic device, according to an embodiment of the present invention.

FIG. 7 is a flow diagram outlining the sequence of a variant of the method for providing a customer access to a unit, according to an embodiment of the present invention.

FIG. 8 is a schematic front elevation view of a bank of multiple storage units, according to another embodiment of the present invention.

FIG. 9 is a schematic representation for procuring an individual compartment in which to store a previously purchased item when returning it, according to an embodiment of the present invention.

FIG. 10 is a schematic representation for a customer physically returning a previously purchased item and its requisite documentation, according to an embodiment of the present invention.

FIG. 11 is a schematic representation of a store staff member retrieving a returned item, according to an embodiment of the present invention.

FIG. 12 is a schematic representation of a store staff member refunding, repairing or exchanging a returned item or rejecting it, according to an embodiment of the present invention.

FIG. 13 is a flowchart of a process the system undertakes for processing a return, according to an embodiment of the present invention.

FIG. 14 is a flowchart of a process the system undertakes for processing an exchange, according to an embodiment of the present invention.

FIG. 15 is a flowchart of a process the system undertakes for processing a repair, according to an embodiment of the present invention.

FIG. 16 is a flowchart of a process the system undertakes for sanitizing an empty compartment, according to an embodiment of the present invention.

FIG. 17 is a flowchart of a process the system undertakes for sanitizing an article in a compartment, according to an embodiment of the present invention.

FIG. 18 is a compartment with two doors, according to an embodiment of the present invention.

### DETAILED DESCRIPTION

### A. Glossary

The term “firmware” includes, but is not limited to, program code and data used to control and manage at least some of the interactions between the various modules of the system.

### GPS-Global Positioning System

The term “hardware” includes, but is not limited to, the compartments and their physical components and also the physical housing for a computer or personal electronic device, as well as display screens, connectors, wiring, circuit boards etc.

### LED-Light Emitting Diode

LTE™ or Long-Term Evolution is a standard for broadband wireless communication.

The term “module” can refer to any component in this invention and to any or all of the features of the invention without limitation. A module may be a software, firmware or hardware module, and may be located in a user's personal electronic device, a server or a bank of compartments. A module may include a processor and a computer readable memory storing computer readable instructions, which, when executed by the processor, provide at least some of the functions of the system.

The term “network” can include both a mobile network and data network without limiting the term's meaning, and includes the use of wireless, e.g. 2G, 3G, 4G, 5G, WiFi™, WiMAX™, Wireless USB (Universal Serial Bus), Zigbee™, Bluetooth™ and satellite, and/or hard wired connections such as local, internet, ADSL (Asymmetrical Digital Subscriber Line), DSL (Digital Subscriber Line), cable modem, T1, T3, fibre, dial-up modem, television cable, and may include connections to flash memory data cards and/or USB memory sticks where appropriate. A network may also mean dedicated connections between computing devices and electronic components, such as buses for intra-chip communications.

NFC—Near field communication

PCR—Polymerase chain reaction, a process used in a COVID-19 test.

The term “personal electronic device” may refer to, for example, a smartphone, a tablet, a smart watch or any other mobile personal communication device that is configured to perform the communications and authentications with the system for facilitating collection, return and exchange of items.

The term “processor” is used to refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). The processor performs at least some of the steps in the flowcharts, whether they are explicitly described as being executed by the processor or whether the execution thereby is implicit due to the steps being described as performed by code or a module. If the processor is comprised of multiple processors, they may be located together or geographically separate from each other. The term includes virtual processors and machine instances as in cloud computing or local virtualization, which are ultimately grounded in physical processors.

QR or Quick Response code—a two-dimensional binary pixelated code that can be scanned in a similar way to a bar code.

RFID—Radio Frequency Identification, used in reference to RFID tags that are used to identify merchandise, for example.

SMS—Short Messaging Service, a service traditionally used for text messaging.

The term “software” includes, but is not limited to, program code that performs some or all of the computations necessary for the system to operate, including, for example, receiving user inputs, communicating between personal electronic devices, the server and the compartments, the reporting of item status, displaying information and providing outputs, etc.

The term “system” when used herein without qualification refers to a system for facilitating the return of items for refund, exchange or repair, and for collecting items. It also refers to locker systems for the purpose of storage and later collection by the same or a different person.

UPC—Universal Product Code, which is a type of bar code.

UV-C is ultraviolet-C radiation having a wavelength of 200-280 nm, and is distinct from UV-A and UV-B.

UVGI—Ultraviolet Germicidal Irradiation

### B. Exemplary Embodiments—Collecting Items

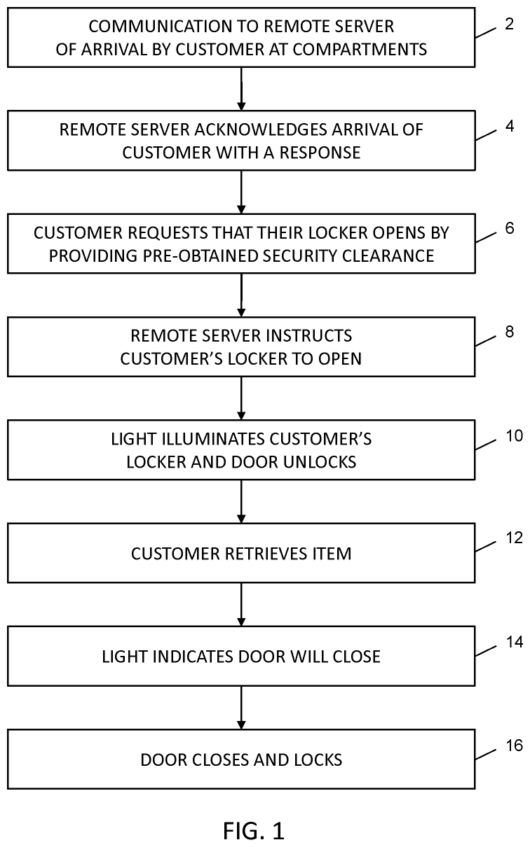

An exemplary method for facilitating collection of items without direct human-to-human interaction that may be used for retail store customer pick up, for example, is described with reference to FIG. 1 through FIG. 6.

Referring to FIG. 1, in step 2 the customer or individual acting on behalf of the customer (both hereinafter referred to as “customer” for brevity) indicates to a remote server that they have arrived at the bank of compartments, which may also be referred to as lockers or units. This may involve the customer scanning a QR code for a web link with a personal electronic device, texting a phone number to the server, or using an NFC protocol to scan the personal electronic device. In other embodiments, other means may be used for informing the server that the customer has arrived at the bank of compartments. In other embodiments, the server and a system of sensors may automatically detect that the customer has arrived at the bank of compartments, e.g. using GPS or facial recognition technology.

Referring to step 4, the remote server acknowledges that the customer has arrived via a response sent to the customer on the customer's personal electronic device. It may be a visual response, an audio response or an audiovisual response, for example. In other embodiments, the response may be sent to an electronic display separate to the personal electronic device, for example, a screen associated with the bank of compartments.

Referring to step 6, the customer then requests that a compartment be opened. Specifically; the compartment to be opened is the compartment in which the item for the customer to collect is located. This may be an implicit request based on the recognition of the customer at the bank of compartments. It may be an explicit request made by the customer via the customer's personal electronic device after the server has acknowledged the presence of the customer at the bank of compartments. It may be an implicit request made as the customer provides any necessary security information to the system to authorize the system to provide access to the compartment.

Referring to step 8, upon successful authorization by the server of the customer, the server then instructs the particular compartment in which the item is located to open. Referring to step 10, the interior light of the particular compartment then illuminates, indicating to the customer or individual which compartment their item is contained in. In relation to this, the door of the compartment unlocks and the door opens automatically. In some cases, the customer retrieves multiple items from the same compartment or from different compartments.

Referring to step 12, the customer then retrieves their item. Referring to step 14, the interior light then indicates to the customer that the compartment will begin to close. For example, the light may dim, or flash. Referring to step 16, the door automatically closes and locks.

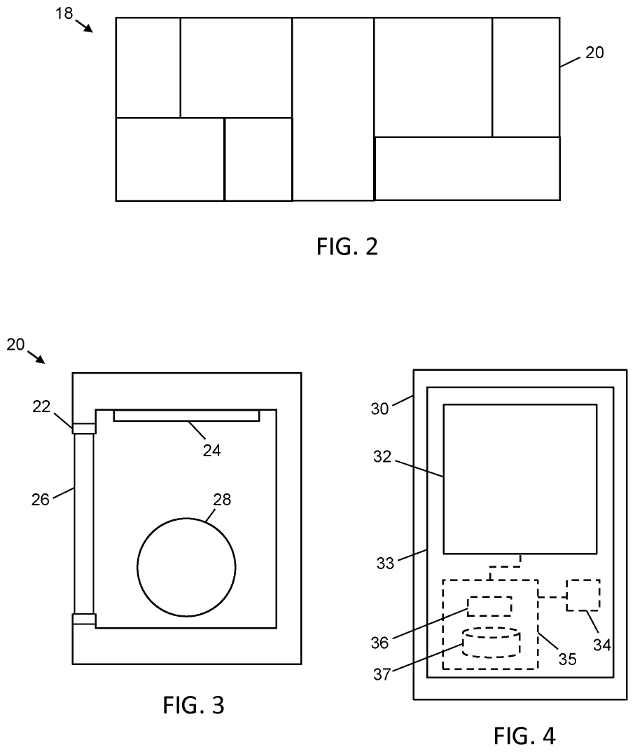

Referring to FIG. 2, a bank of compartments 18 is provided. Bank of compartments 18 consists of a plurality of individual compartments 20. Banks of compartments 18 may be modular. Individual compartments 20 may be either uniform in size or have one or more different sizes or shapes, and in some embodiments they may all have different sizes and/or shapes. The compartment does not have restrictions on how large or small it may be. It is of a highly modular nature, which allows for it to be placed as a standalone storage unit or to be a part of a large bank of compartments. Since the compartment can be of any given size, the bank which it may be integrated into may have compartments of all sizes, allowing for a range of storage capabilities.

Referring to FIG. 3, which is a side sectional view of an interior of an individual compartment 20, it shows that each individual compartment has a door 22. An interior light 24 is mounted within compartment 20. Compartment door 22 may have a transparent or translucent panel 26 through which a customer may view an item 28 inside compartment 20 when interior light 24 is illuminated. Alternatively, the panel may be opaque, and the interior light may be placed to be visible outside of the panel. For example, it may be a light that is mounted on the outside of the compartment 20, either on the body of the compartment or on the door 22 of the compartment. In this case, the light is used to identify the compartment to the customer rather than to illuminate the contents of the compartment.

Referring to FIG. 4, the system includes or interacts with a personal electronic device 30, which may be a tablet, smartphone or smart watch, for example, or any other electronic device that provides the necessary equivalent functionality for communicating with the server. Upon the customer communicating with the server via personal electronic device 30, a temporary communication interface 32 is provided to personal electronic device 30 on screen 33 of the personal electronic device. Temporary communication interface 32 may be a link in a web browser, an application, or other temporary communication interface. The customer requests or initiates communication with the server, and the server establishes the communication link with the customer.

The personal electronic device 30 includes one or more processors 34 which are operably connected to computer readable memory 35 included in the personal electronic device. The personal electronic device 30 includes computer readable instructions 36 (e.g. an application, which may be a browser) stored in the memory 35 and computer readable data 37, also stored in the memory.

Some or all of the computer readable instructions 36 and computer readable data 37 provide at least some of the functionality of the system when executed or read by the processor(s) 34. Computer readable instructions may be broken down into blocks of code or modules. The memory 35 may be divided into one or more constituent memories, of the same or different types. The personal electronic device 30 includes the display screen 33, operably connected to the processor(s) 34. The display screen 33 may be a traditional screen, a touch screen, an electronic ink display or any other technological device for displaying information.

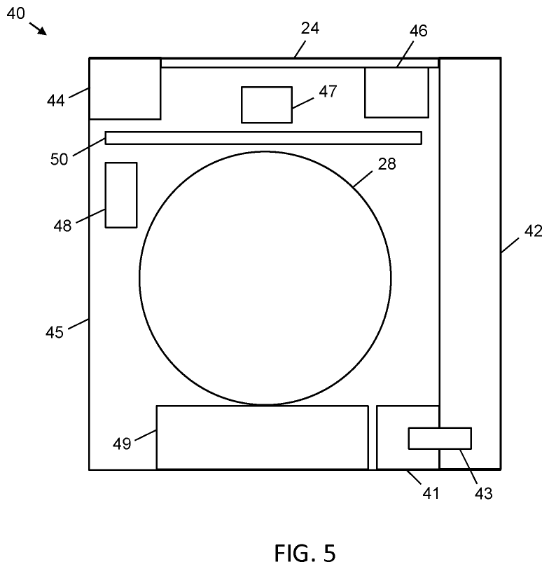

Referring to FIG. 5, another compartment 40 is shown from the front with its door 42 in the open position to the right side, revealing interior components. A locking mechanism 44 is mounted to the compartment wall 45. A communication interface module 48 is located at the rear of compartment 40, though the exact position is arbitrary. A motor 41 is provided which controls the opening and closing of compartment door 42. A linkage arm 43 connects motor 41 with compartment door 42. The interior light 24, as previously described, is mounted within compartment 40. Alternatively, if the compartment door 42 is solid, the interior light may be mounted in such a way that it is visible outside of the compartment 40.

An optional camera 47 may be mounted within compartment 40, and it may be a still camera, infra-red camera or a video camera depending on the embodiment. Camera 47 may be used for many tasks such as identifying that the correct item 28 has been placed in the compartment, that an item has been removed, capturing an image of a person removing an item, facial recognition, and others. An optional load cell 49 may be used to identify when item 28 has been removed from compartment 40, by detecting a change in weight to which the upper surface of the load cell is subjected. In a retail pick up application, load cell 49 can also identify if the wrong item is placed in the compartment, by determining a discrepancy between an actual weight of an item and the weight that the item purchased should have. An audio speaker 46 may be included in or on the outside of the compartment for any audio needs such as remote support and audio cues. Other sensors may be included inside or outside of the compartment 40, such as ultrasound, infrared, passive infrared, radar, and thermal sensors.

In some embodiments, one or more of the sensors are shared between multiple compartments, particularly if they are mounted on the outside of the compartments. For example, the camera 47 may be outside the compartment and may be used for multiple compartments within the same bank of compartments. The same may apply to the audio speaker 46, for example.

Still referring to FIG. 5, compartment 40 may include a disinfection system to further enhance the touchless aspects of the system. For example, ultraviolet germicidal irradiation (UVGI) may be used as a disinfection method using short-wavelength ultraviolet (ultraviolet C or UV-C) light to kill or inactivate microorganisms and viruses by destroying nucleic acids and disrupting their DNA, or by damaging other parts of them, leaving them unable to perform vital cellular or viral functions. In this example, a UV-C light source 50, such as an LED or array of LEDs may be installed in the compartment and switched on as and when necessary to sanitize the interior of the compartment and/or the contents of the compartment. It is not effective to use regular ultraviolet lighting as the wavelengths emitted by such lighting generally do not extend to the UV-C range, and the wavelengths that are emitted (UV-A and UV-B) are relatively inefficient in killing the COVID-19 virus. For UV sterilization, only UV-C (100-280 nm) has high enough energy to effectively kill microorganisms.

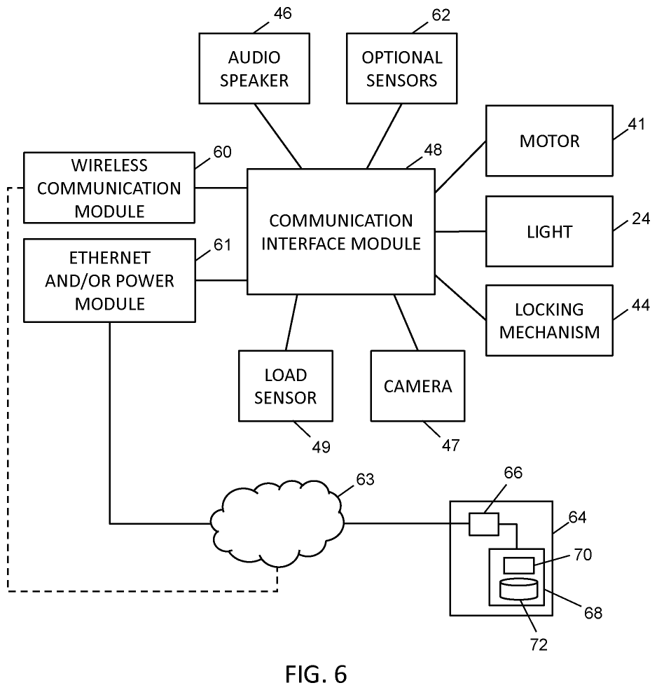

In order for all components of the system to function in a coordinated fashion, there is communication between the various components and the server. Referring to FIG. 6, a schematic drawing of communication interface module 48 and its connections is illustrated. The connections to components such as light 24, motor 41, locking mechanism 44, load cell 49, camera 47, audio speaker 46 and any other optional sensors 62 are provided here to give insight to non-limiting optional configurations. The communication interface module 48 is connected via the internet 63 to the remote server 64 initially mentioned with respect to FIG. 1 via the wireless communication module 60 solely, via the Ethernet and/or power module 61 solely, or via a combination of the wireless communication module and the Ethernet and/or power module. Wireless communication module 60 may operate using any or any combination of Wi-Fi™, Bluetooth™, NFC, 5G, LTE™, or other radio communication protocol. The Ethernet and/or power module 61 may provide power to the communication interface module over Ethernet or from a traditional wall plug. Communication interface module 48 has sufficient processing capabilities along with all requisite communication interfaces such as the wireless communication module 60 and the Ethernet and/or power module 61 for any particular configuration.

The server 64 includes one or more processors 66 which are operably connected to computer readable memory 68 included in the server. The server 64 includes computer readable instructions 70 (e.g. an application) stored in the memory 68 and computer readable data 72, also stored in the memory. Some or all of the computer readable instructions 70 and computer readable data 72 provide at least some of the functionality of the system when executed or read by the processor(s) 66. Computer readable instructions may be broken down into blocks of code or modules. The memory 68 may be divided into one or more constituent memories, of the same or different types. The server 64 may include multiple constituent servers either co-located or geographically separate from each other.

The method described above facilitates collection of items without human interaction. If a touchless experience is desired to reduce the need for sanitation and reduce the likelihood of spreading COVID-19 or other communicable diseases, the method may be configured to automatically open and close the compartment door based on triggering events, such as expiry of a predetermined time interval. The storage unit or compartment door 42 is attached to an arm 43, which is attached to a motor 41. This motor 41 extends and retracts the arm 43, which causes the door 42 to open and close without anyone physically touching the door. Due to the nature of the motor 41, controlling the door opening and closing, there is also an inherent level of security provided by the motor. To further increase security, a locking mechanism 44 may be used in conjunction with the door. The locking mechanism 44 may be of a mechanical, electromagnetic, or another nature.

As the storage unit or compartment may have a door that is opaque, translucent or clear, this allows for aesthetic considerations to be had as well as for providing security. The compartment may be made from any material to provide any desired characteristics to the compartment. For example, the interior of the compartment may be lined at least in part with one or more aluminum or other reflectors that reflect the UV-C light around the compartment and around the item within the compartment. As another example, the walls of the compartment may be thick or thermally insulating in order to maintain hot foods at an elevated temperature compared to room temperature. The compartment may have one or more doors with one or more motors per door or it may have a single motor that controls all doors. In one embodiment, the greater than one door includes a configuration with a front-opening door and a rear-opening door. With both doors, the locker unit can be placed in walls allowing for store staff to place items from inside the retail establishment while customers collect their items from outside the retail establishment. This allows customers to interact with the store outside of normal business hours. Additionally, the rear-opening door may allow units to be filled autonomously by robots.

The compartment may also have cameras covering the interior and/or exterior for facial recognition, security, or any other use that a camera would be considered for. Access to the compartment may include prior facial recognition or other biometrics such as, but not limited to, fingerprints. Biometrics, such as fingerprints and others, may be detected by the customer's personal electronic device, for example. The compartment may also use NFC technology to grant access. The compartment may have other sensors 62 within it such as temperature and humidity sensors etc. This allows for better security, a guarantee that items have remained in an appropriate environment, and other advanced capabilities depending on which sensors are included in any given configuration of the compartment. It is also conceivable that the compartment have integrated, or be coupled with, advanced detectors such as mass spectrometers, gas chromatographs or other advanced detection devices which may detect unsafe materials in the compartment and provide advanced warning to a customer or staff member who may desire to open the compartment. Unsafe materials may include corrosive, poisonous, flammable and explosive substances, for example.

The motor 41, is controlled by the communication interface module 48, which processes signals that determine when the door 42 should open and when the door should close. Any included sensors are also connected to this controlling unit 48, either via wired or wireless connections. The compartment opens and closes as instructed by the communication interface module 48, which is instructed by the server via the personal electronic device on behalf of the server. The communication interface module 48 may control one or more compartments. It may also communicate with other communication interface modules 48 in other compartments.

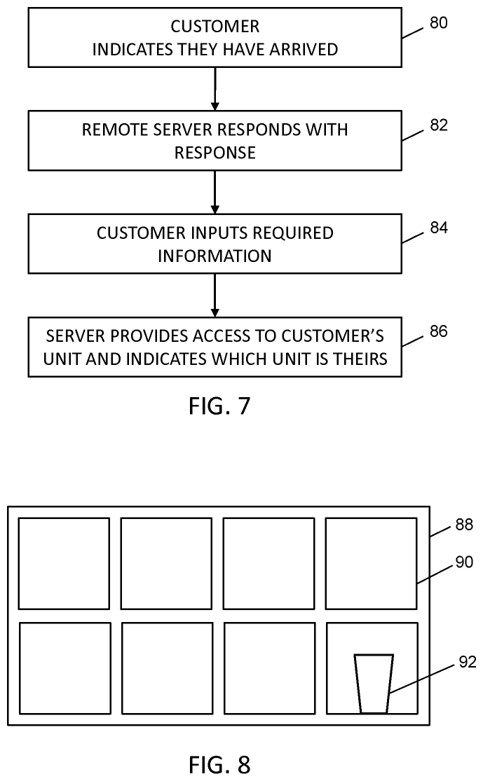

In a variation of the method, a simplified version that may be used for coffee pick up is described with reference to FIG. 7 and FIG. 8. This variant was developed for use where fear of theft is not a problem, but there is still a desire to limit one's interaction with other people. The present method can be used in such cases to expedite the collection of the coffee or take-out meal without any interaction with other people.

Referring to FIG. 7, step 80, the customer indicates to the remote server that they have arrived at the compartments. This may be accomplished via a QR code for a web link, texting of a phone number, NFC, or another means. Referring to step 82, the remote server acknowledges that the customer has arrived via a response to the customer's personal electronic device. Referring to step 84, the customer provides any necessary security or other information to the system. Referring to step 86, upon successful authorization of the customer, the remote server then informs the customer, via the customer's personal electronic device, of the compartment from which their coffee or take-out meal can be collected.

Referring to FIG. 8, a bank of compartments 88 is provided, which has a plurality of individual compartments 90 and may be modular. Individual compartments 90 may be either uniform in size or have different sizes. Item 92 is placed in a selected one of the individual compartments 90 by a staff member, to await pickup. Compartments may be identified by numbers, letters or lights that are activated by the server as previously described. In some embodiments, there may be no doors on the compartments.

### C. Exemplary Embodiments—Returning Items

Returns of previously purchased items may be accomplished in four stages, including: stage 1, procure an individual compartment from the bank of compartments; stage 2, placement of a previously purchased item into the individual compartment by the customer; stage 3, a store staff member collecting the item; and stage 4, issue of refund or exchange, or denial of return or exchange and the item is placed back into the compartment for the customer to collect.

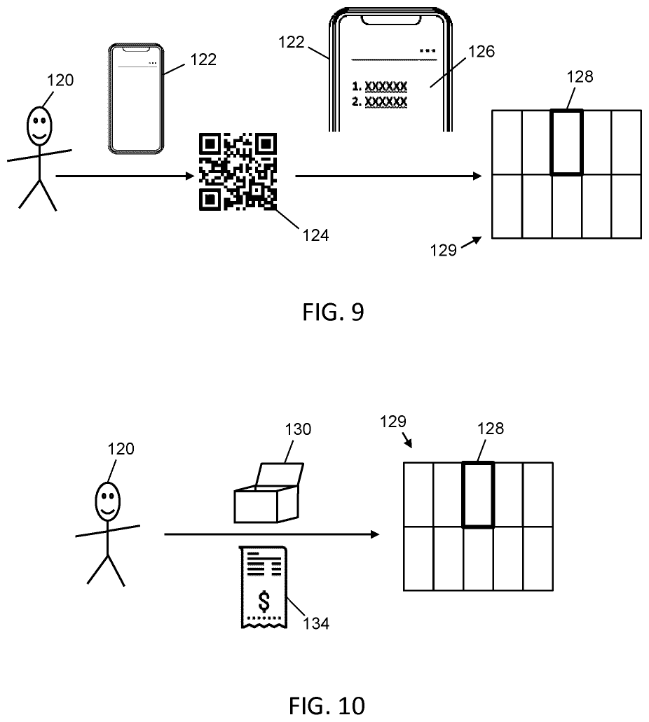

In the first stage for the return of a previously purchased item, referred to in FIG. 9, a customer 120 who wants to return their previously purchased item to a retailer initiates the first step of a contactless return. The result of this first stage is to procure an individual compartment 128 from the bank of compartments 129 in which to place the previously purchased item.

In one embodiment the customer 120 may need to be within a set or predetermined physical proximity to an item return location to initiate the return of their previously purchased item. Such proximity may be determined through location tracking software in the personal electronic device 122 used by the customer, for example, or by the customer tapping their personal electronic device on a detector at or near the return location. An example of a suitable physical proximity may be that the customer is in the same room as the bank of compartments, or is within a set distance such as 5 m or 10 m.

The customer 120 uses their personal electronic device 122 to connect to the server in order to return the previously purchased item by scanning the scannable entity 124. The scannable entity 124 may include any digitally recognizable code, including but not limited to a QR code, UPC code, or RFID tag that a store may use to identify individual items for purchase and return. The personal electronic device 122 or other scanning device may be used to scan the scannable entity 124. Scannable entity 124 includes a link which directs the customer 120 through the personal electronic device 122 to the temporary communication interface 126 which is unique to the user session. Each connection to the server for returns is unique to the user session with the server. The user-server session for returns includes the temporary communication interface 126 displayed on the personal electronic device 122.

A user session is created through the temporary communication interface 126 to allow the customer to interact with the server and provide information as required for the return, including but not limited to the reason for the return. The user session on the temporary communication interface for each return transaction continues until the return transaction is complete, abandoned or saved to be completed later.

The temporary communication interface 126 may be presented on a webpage, as an application, or on other mediums using which a customer can interact with the temporary communication interface. The temporary communication interface 126 presents customer 120 with a user interface representing a bank of compartments 129. In some embodiments, the temporary communication interface 126 allows the user to select an individual compartment from the bank of compartments 129 in which to store their previously purchased item. In one embodiment the customer may select an individual compartment for return of the previously purchased item from the bank of compartments appropriate for the previously purchased item to be returned based on, but not limited to, such features as the size, shape, and value of the item to be returned. In another embodiment the selection of an individual compartment for return of the previously purchased item from the bank of compartments appropriate for the previously purchased item to be returned may be made by the temporary communication interface based on, but not limited to, such features as the size, shape, and value of the item to be returned. This requires the server previously determining or being informed of the item or aspects of the item to be returned.

Once the customer 120, has selected an individual compartment 128 from the bank of compartments 129 for return of their previously purchased item, represented within the temporary communication interface 126, the individual compartment can now be used and accessed by only that customer 120, and cannot be accessed by any other customer or any third party.

The individual compartment 128 may be a secure location for the customer 120 to leave their previously purchased item to be returned. Physical access to bank of compartments 129 may be at fixed hours as set by the customer through the temporary communication interface 126, as set by the store, or they may be available up to 24 hours a day, 7 days a week. The customer may return their previously purchased item during these hours, which may include return outside of regular business hours. In one embodiment the hours to physically access the bank of compartments may be altered by the customer, or alternatively the store. In some embodiments, the customer is given a particular time slot or choice of time slots with which the item may be returned. For example, it may be a 5-minute time slot or a 10-minute time slot, and it may be scheduled to avoid the customer being at the bank of compartments at the same time as another customer or a staff member. If the bank of compartments is large enough, multiple customers may be directed at the same time to compartments that are sufficiently separate from one another in an attempt to reduce the spread of COVID-19 or other infections. Such separation between the compartments that are used by customers and/or staff members at the same time may be 2 m or 3 m apart from each other, for example. The customer is then able to choose which time slot to use for the return by selecting it on the temporary communication interface 126.

Once a customer 120 has procured an individual compartment 128 and all requisite conditions associated with procuring the individual compartment as presented by the individual compartment procurement interface 126, the individual compartment automatically moves to an open state, and then the customer begins the second stage of a contactless return.

The second stage of the return, referred to in FIG. 10, includes the customer 120) placing their previously purchased item 130 and requisite documentation 134 such as a receipt or other documentation into their procured individual compartment 128 in the bank of compartments 129. Alternatively, the customer may use the temporary communication interface 126 (FIG. 9) to upload their previously purchased item requisite documentation. The customer 120 places their previously purchased item 130 and requisite documentation 134 into compartment 128 such that their procured individual compartment then moves to a closed state.

In some embodiments the individual compartment 128 opens automatically, and also closes automatically, without the customer needing to touch the compartment or its door. The automatic, touchless closing of the individual compartment in the bank of compartments 129 is initiated by the customer interacting with the temporary communication interface 126 to indicate that their item has been placed in the individual compartment. In another embodiment, a camera and/or other sensors may be used to detect that the item and optionally the documentation have been placed in the compartment. In another embodiment, the closing is initiated by the customer stepping away from the compartment. In another embodiment the individual compartment closing can be initiated by a timed session, such session may be set or extended by the customer 120 by interacting with the still existing temporary communication interface 126. The system may include a failsafe timer in which the individual compartment open state can no longer be extended or set by the customer and will close to ensure that the individual compartment is not kept open perpetually:

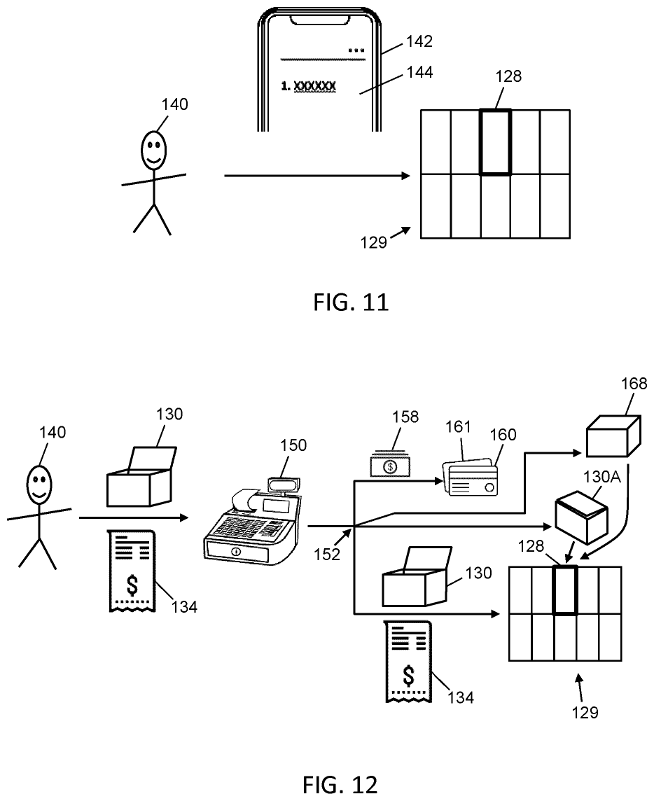

In a third stage of the return, referred to in FIG. 11, a member 140 of store staff connects to the server with a server connection, using, for example a personal electronic device 142, or a store device or other computer. Each connection to the server for returns may include a unique session with the server. The store staff server session for returns includes a secure communication interface 144. Such communication interface may be a temporary communication interface. Store staff member 140 uses the communication interface 144 to locate the occupied individual compartment 128 in the bank of compartments 129.

The store staff member 140 records, through the communication interface 144, that they are going to commit to facilitating the return of the contents of the individual compartment 128. Upon committing to facilitating the return of the individual compartment contents, the associated individual compartment 128 in the bank of compartments 129 automatically moves to an open state as prescribed by communication interface 144.

In an alternate embodiment the store staff member 140 may delay or postpone facilitating the return of the contents of the individual compartment 128 after the door has opened. This may be done through the temporary communication interface 144, wherein the associated individual compartment 128 in the bank of compartments 129 automatically returns to a closed state as prescribed by communication interface 144.

The fourth stage of the return process is shown in FIG. 12. With the individual compartment 128 in an open state, the store staff member 140 collects the previously purchased item 130 and requisite documentation 134 which the customer placed in the individual compartment as described in stage 2 with respect FIG. 10. Store staff member 140 evaluates the previously purchased item 130 and requisite documentation 134, and may interact with point-of-sale system 150 to facilitate the return of the previously purchased item and requisite documentation.

Decision point 152 represents a fork in the method contingent on the validity of the return of the previously purchased item 130 and requisite documentation 134. If the previously purchased item 130 and requisite documentation 134 are determined to be a valid return by store staff member 140, the staff member uses the point-of-sale system 150 to return the monetary funds 158 that the customer had originally used to purchase the previously purchased item. The staff member 140 refunds the amount of monetary funds 158 to the same monetary instrument 160 that was used to make the original purchase. If the same monetary instrument is unavailable, then another monetary instrument 161 may be used. For example, a credit note may be issued. Note that the conditions for a return to be valid, as determined by the store staff, may be different to the steps needed to be made by the customer to effect a valid deposit of the returned item in the compartment 128.

If store staff member 140 determines the previously purchased item 130 to be ineligible for return at decision point 152, then store staff member 140 returns the previously purchased item 130 and requisite documentation 134 into the individual compartment 128 in the bank of compartments 129. The door of the individual compartment automatically moves to a closed state and the customer is notified to collect their item within a prescribed amount of time.

If the previously purchased item 130 is intended to be an exchange and not a return, then at decision point 152, if store staff member 140 has determined that the previously purchased item 130 is eligible for an exchange, then the store staff member places a new item 168 into the individual compartment 128 of the bank of compartments 129 and the door automatically moves to a closed state. The customer 130 is then notified that they can collect their new item 168 within a prescribed amount of time, from the individual compartment 128 in the bank of compartments 129. The notification of the requirement for the customer 130 to collect their item may be executed through an SMS communication, through an application interface, through a webpage update, or through other means of communication.

If the previously purchased item 130 is intended to be an exchange and not a return, then at decision point 152, if store staff member 140 has determined that the previously purchased item 130 is ineligible for an exchange, then the store staff member places the previously purchased item 130 back into the individual compartment 128 of the bank of compartments 129 and the door automatically moves to a closed state. The customer is then notified that they can collect their previously purchased item 130 within a prescribed amount of time from the individual compartment 128 in the bank of compartments 129.

In another embodiment, the previously purchased item 130 may be returned for repair. At decision point 152, if store staff member 140 has determined that the previously purchased item 130 is eligible for repair, for example under a warranty, then after repair the store staff member places the repaired item 130A back into the individual compartment 128 of the bank of compartments 129 and the door automatically moves to a closed state. If the same compartment is not available, then another compartment is used. The customer 120 is then notified that they can collect their repaired item within a prescribed amount of time from the corresponding individual compartment in the bank of compartments 129.

If the previously purchased item 130 is returned for repair, but at decision point 152 store staff member 140 determines that the previously purchased item 130 is ineligible for repair, then the store staff member places the previously purchased item 130 back into the individual compartment 128 of the bank of compartments 129 and the door automatically moves to a closed state. The customer is the notified that they can collect their previously purchased item 130 within a prescribed amount of time from the individual compartment 128 in the bank of compartments 129.

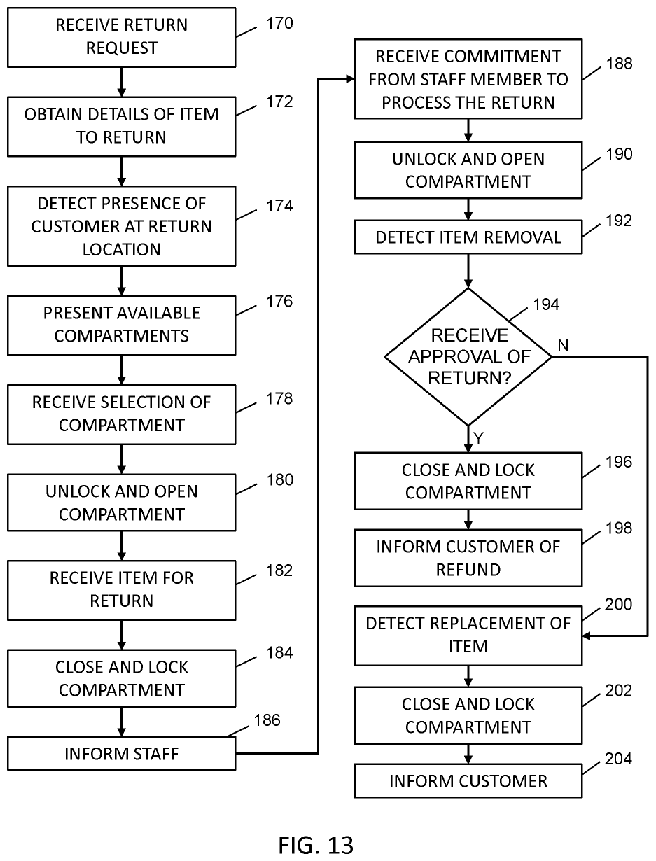

FIG. 13 shows an exemplary process that the system undertakes for processing a return. In step 170, the system receives a request from a customer to return a purchased item. This may be an implied request based on the customer requesting that a communication link be established with the server. In step 172, which may alternately be performed later in the process, the system obtains details about the item that the customer would like to return, such as the item name, stock number, bar code, etc. In step 174, the system detects the presence of the customer at a return location, for example, at a bank of compartments in a store. There may be a time gap between when the system receives the request and when it detects the presence of the customer at the return location. This depends on where the customer is when the request to return the item is made. For example, the customer may already be at the return location or may be at home. The system may detect the presence of the customer at the location by way of an input from the customer to the system, or by way of the system automatically detecting the location of the customer's personal electronic device. In some embodiments, steps 172 and 174 are switched. In other embodiments, step 172 is omitted and instead the documentation with details of the item is placed in the compartment with the item.

In step 176, the system presents to the customer a selection of the available compartments at the return location, i.e. the compartments in a bank of compartments that are empty, or empty and sanitized. If the system is aware at this point of the size of the item to be returned, then the available compartments are limited to those that are large enough to accommodate the item. Also, the compartments may be offered at particular time slots, or a set of available time slots may be offered for the selected compartment. In step 178, the system receives a selection from the customer of one of the presented available compartments. Again, this step may be optional if only one of the compartments is available or suitable. In step 180, the system unlocks and opens the selected compartment. In step 182, the system receives the item to be returned in the unlocked and opened compartment. In step 184, the system closes and locks the compartment in which the item for return has been placed.

In step 186, the system then informs one or more members of staff that an item has been returned, and informs them of the compartment in which the item for return is located. This may be immediately upon the compartment being locked or after a delay, during which the item may be sanitized. In step 188, the system receives an input from one of the staff members indicating a commitment that they are going to process the return. In step 190, the system unlocks and opens the compartment. In step 192, the system detects that the item for return has been removed from the compartment. This may be via a load sensor indicating a drop in sensed weight to zero, or via the receipt of an input from the staff member indicating that they have removed the item from the compartment.

In step 194, the system determines whether it has received an approval for the return, or whether the return has been rejected. This may take place by the system receiving an input from the staff member that indicates that the item has been rejected or approved for return. This input may be provided to the system by the staff member selecting a corresponding option on their personal electronic device. In step 196, the system then closes and locks the compartment. In step 198, the system informs the customer that the return has been accepted and that they have or will get a refund. Depending on how the point-of-sales equipment is integrated with the system, the system may also process the refund to the customer. In other embodiments, the staff member processes the return of funds.

If in step 194, the system determines that the request for returning the item has been rejected by the staff member, then the process moves to step 200, in which the system detects the replacement of the item back into the compartment. This may be via a load sensor indicating an increase in sensed weight from zero to about the expected weight of the item including its packaging, or via the receipt of an input from the staff member indicating that they have replaced the item in the compartment. In step 202, the system closes and locks the compartment. In step 204, the system informs the customer that the request for return has been rejected and that they should come back to the bank of compartments to collect their item. The system then fulfils the necessary steps in order for the customer to collect the repaired item according to FIG. 1.

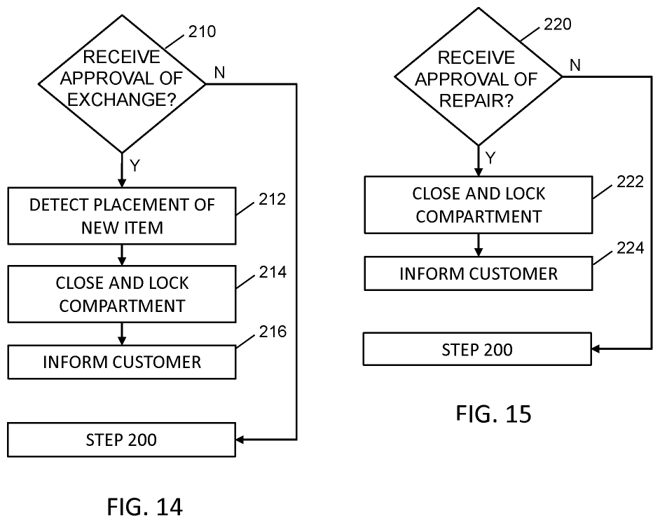

FIG. 14 is a flowchart of a process the system undertakes for processing an exchange. Many of the steps are similar to steps 170-192 of FIG. 13, except that they refer to an exchange of an item rather than an item to be returned for a refund. After the system detects the removal of the item from the compartment by a staff member, in step 192, the process moves to step 210 of FIG. 14.

In step 210, the system determines whether it has received an approval for the exchange, or whether the request has been rejected. This may take place by the system receiving an input from the staff member that indicates that the item has been rejected or approved for exchange. This input may be provided to the system by the staff member selecting a corresponding option on their personal electronic device. If approved, then in step 212, the system detects the replacement of a new item back into the compartment. The item may be the same kind of item as the one that was returned, or it may be an equivalent one or even a different product. If different, then the member of staff processes the difference in value, if any, by refunding or charging the customer accordingly. The system may detect the placement of the new item via a load sensor indicating an increase in sensed weight from zero to about the expected weight of the new item including its packaging, or via the receipt of an input from the staff member indicating that they have placed a new item in the compartment. In step 214, the system closes and locks the compartment. In step 216, the system informs the customer that the request for exchange has been approved and that they should come back to the bank of compartments to collect their new or exchange item. The system then fulfils the necessary steps in order for the customer to collect the new item according to FIG. 1.

If in step 210 the system determines that the request for exchanging the item has been rejected by the staff member, then the process moves to step 200 of FIG. 13.

FIG. 15 is a flowchart of a process the system undertakes for processing a repair. Many of the steps are similar to steps 170-192 of FIG. 13, except that they refer to a repair of an item rather than an item to be returned for a refund. After the system detects the removal of the item from the compartment by a staff member, in step 192, the process moves to step 220 of FIG. 15.

In step 220, the system determines whether it has received an approval for the repair, or whether the request has been rejected. This may take place by the system receiving an input from the staff member that indicates that the item has been rejected or approved for repair. This input may be provided to the system by the staff member selecting a corresponding option on their personal electronic device. If approved, then in step 222, the system closes and locks the compartment. In step 224, the system informs the customer that the request for repair has been approved and that they should come back to the bank of compartments at a particular time and/or day to collect their repaired item. In other embodiments, the system informs the customer that the request for repair has been approved and that they will be informed by the system at a later time and/or date when to collect it. This latter message may be sent by the system after the system has received a notification from a staff member that the item has been repaired and actually placed into a compartment. After the system has received the repaired item in one of its compartments, the system fulfils the necessary steps in order for the customer to collect the repaired item according to FIG. 1.

If in step 220, the system determines that the request for repairing the item has been rejected by the staff member, then the process moves to step 200 of FIG. 13.

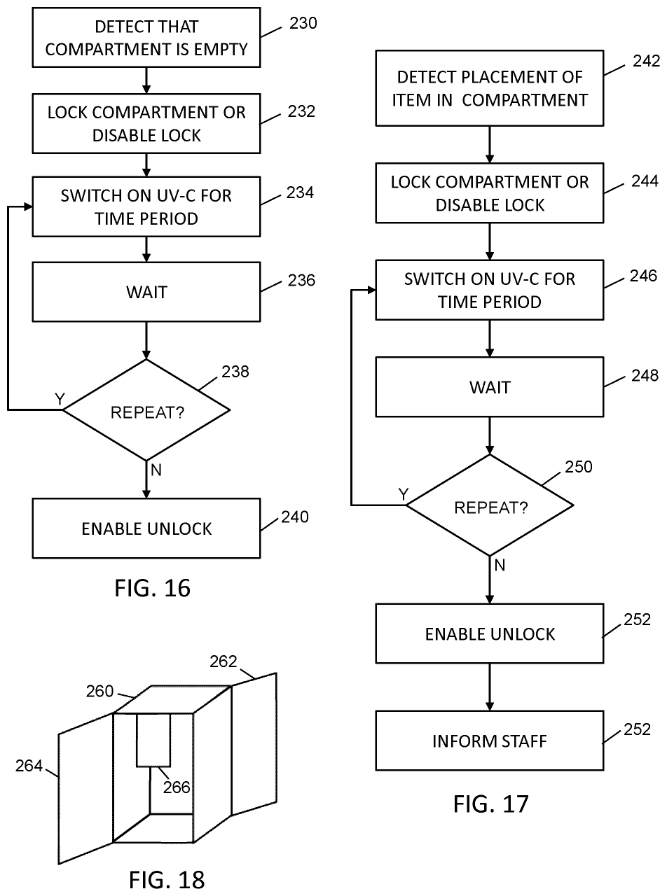

FIG. 16 is a flowchart of a process the system undertakes for sanitizing an empty compartment. In step 230, the system detects that a compartment is empty. In step 232, the system locks the compartment if it is not already locked. In some embodiments, the lock may be additionally disabled so that it cannot be unlocked by an overriding command or physical action. This is to avoid the door inadvertently being opened by a customer or a staff member while the UV-C lights are on. In other embodiments, the compartment is equipped with a safety interlock that cuts power to the UV-C light when the door is opened.

In step 234, the system switches on the UV-C lights in the empty compartment for a predetermined time period. For example, the period may be 30 minute or 1 hour, or according to durations otherwise defined in sanitation guidelines that are available to one skilled in the art, and it will depend on the strength of the UV-C LEDs. A skilled person can readily select the output power of the UV-C LEDs and their illumination durations for a particular size of compartment to provide sufficient virus and other microbe kill rates, based on published disinfection rate data. This may be long enough for the compartment to be sanitized, or, if the UV-C lighting is switched on for longer, the compartment may be sterilized. For example, in some use-cases. PCR tests may be provided and submitted using the bank of compartments, or collection kits and other samples that contain pathological life forms may be provided using the compartments. In these cases, the exterior of the contents placed in the compartments may be sterilized, and/or the compartments may also be sterilized after use, when empty. In other embodiments, it may be desired to sanitize or sterilize the compartment in two or more steps. In step 236, the system optionally waits, after the UV-C lights have been switched off, for a predetermined period of time. In step 238, the system determines whether to repeat the switching on of the UV-C lights, and if so, the process reverts to step 234. If the system determines in step 238 that further lighting is not necessary, then in step 240 the system enables the lock on the compartment such that it may be operated. i.e. unlocked, for use by a staff member or a customer.

FIG. 17 is a flowchart of a process the system undertakes for sanitizing an article in a compartment. In step 242, the system detects the placement of an item in the compartment, either by way of being informed of such or by detecting a weight on the load sensor. In step 244, the system locks the compartment if it is not already locked and/or additionally disables it.

In step 246, the system switches on the UV-C lights in the occupied compartment for a predetermined time period. This may be long enough for the compartment and contents to be sanitized, or, if the UV-C lighting is switched on for longer, the compartment and contents may be sterilized. This may depend on the shape of the item and whether all parts of it are exposed to the UV-C lighting. In some embodiments, it may be desired to sanitize or sterilize the compartment in two or more steps. In step 248, the system optionally waits, after the UV-C lights have been switched off, for a predetermined period of time. In step 250, the system determines whether to repeat the switching on of the UV-C lights, and if so, the process reverts to step 246. If the system determines in step 250 that further lighting is not necessary, then in step 252 the system enables the lock on the compartment such that it may be operated, i.e. unlocked, for use by a staff member or a customer. In step 252, a staff member is then informed. For example, the staff member may be informed by the system that an item has been returned for refund, exchange or repair and that it has been sterilized or sanitized.

FIG. 18 shows a compartment 260 with a front-opening door 262 and a rear-opening door 264. Another aspect of the system is that is may be configured for robots to be used for pick-up, delivery, or both. In some embodiments, the doors of the compartments to be used are configured to unlock and open in the presence of the robot, which allows the robot to retrieve an item from the compartment or deposit an item in the compartment. A robot may work on one side of the bank of compartments while the general public are allowed access to the other side. In some embodiments, robots may access both sides of the bank of compartments. FIG. 18 shows a sensor 266 or other apparatus that a robot may use to fill the individual compartment 260. For example, the sensor may detect the presence of the robot in the vicinity (e.g. within 0.5 m or 1 m) of the particular compartment and unlock and open the compartment in response. When the robot has collected the item, or placed one in the compartment and moved away, the sensor 266 detects the absence of the robot in the vicinity of the compartment and triggers the closing and locking of the door. In embodiments where robots are used, there may be a reduced risk of theft and so there may be no locks in compartments in these locations.

### D. Variations of the Embodiments

While a method has been described for facilitating collection, return and exchange of items without human interaction that was developed for retail store pickup, return and exchange, it has wider application. For example, it may be used for lockers in change rooms or at airports, or in other public locations where the public are not allowed to take certain of their personal belongings into a particular space. It applies equally to storage lockers or storage units.

The system may also be used for the collection of medication from a pharmacy, picking up collection containers and dropping samples (e.g. urine) off for pick up by a medical laboratory, or for services such as key cutting, skate sharpening, shoe repair, tailoring, posting, engraving, picture framing etc. The system is useful for anything that requires a remote drop off and pick up of an item. For example, in relation to FIG. 12, the previously purchased item 130 may be for servicing such as key cutting, skate sharpening, etc. In another embodiment, the previously purchased item 130 is a medical sample such as a urine test, PCR test, etc. The sample is deposited into the individual compartment 128. The store staff in this case may be medical professionals who then collect it for processing in a medical analysis laboratory, or they may be delivery agents that collect the samples to take them to a laboratory or other similar company. The results of the testing are then communicated from the laboratory to the patient via usual channels, such as telephone or email notification to access a website, or to the patients doctor. In another embodiment, the previously purchased item 130 is a prescription. In this use case the previously purchased item 130 is a prescription that is placed into the individual compartment 128 by the customer. The store staff fill the prescription, place it in the individual compartment, or another one, and a link is then communicated to the customer. This link provides the customer with access to the compartment to collect their prescribed medication. In other embodiments, the collection of prescription medication may be provided electronically to the pharmacy, and then the system follows the steps in FIG. 1 to allow the customer to collect the medication.

In some embodiments, the item may be quarantined in the compartment for an extended period without any application of UV-C radiation, such as a day, to allow microorganisms and viruses to decompose naturally. In other embodiments, a combination of quarantine and UV-C irradiation may be used.

Features from any of the embodiments may be combined with features from any of the other embodiments to form another embodiment within the scope of the invention.

Embodiments, depending on their configuration, may exhibit all or fewer than all of the advantages described herein. Other advantages not mentioned may be present in one or more of the embodiments.

The temporary communication interface may exist in a single session, or it may be spread over multiple sessions, for example if the customer decides to pause the completion of the return.

In this document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

Throughout the description, specific details have been set forth in order to provide a more thorough understanding of the invention. However, the invention may be practised without these particulars. In other instances, well known elements have not been shown or described in detail and repetitions of steps and features have been omitted to avoid unnecessarily obscuring the invention. It will be clear to one having skill in the art that further variations to the specific details disclosed herein can be made, resulting in other embodiments that are within the scope of the invention disclosed. Steps in the flowcharts may be removed or other steps added without altering the main outcome of the process. Two or more steps in the flowcharts may be performed in a different order, other steps may be added, or one or more may be removed without altering the main function of the invention. Flowcharts from different figures may be combined in different ways. Modules may be divided into constituent modules or combined into larger modules. All parameters, materials, quantities, durations, measurements, proportions and configurations described herein are examples only and may be changed depending on the specific embodiment.

The scope of the claims should not be limited by the illustrated embodiments set forth as examples, but should be given the broadest interpretation consistent with the description as a whole.

What is claimed is:

1. A method for facilitating a return by a customer of an item that has been purchased, comprising:
establishing, by a server, a communication link with the customer;
determining, by the server, that the customer is present at a bank of compartments;
opening, by instruction from the server, a compartment of the bank of compartments;
determining, by the server, that the item has been placed in the compartment;
instructing, by the server, the compartment to close;
informing, by the server, a staff member that the item has been returned; and
identifying, by the server, the compartment to the staff member.

2. The method of claim 1, wherein the communication link is established via a temporary communication interface on a personal electronic device of the customer, the method further comprising:
providing, by the server, the temporary communication interface;
receiving, by the server, an authentication input from the customer via the temporary communication interface; and
instructing, by the server, a light associated with the compartment to illuminate.

3. The method of claim 1, further comprising receiving, by the server, a request to return the item, wherein:
the request is received via the temporary communication interface; and
the return is for a refund, an exchange or a repair.

4. The method of claim 1, further comprising:
unlocking the compartment prior to opening the compartment; and
locking the compartment after instructing the compartment to close.

5. The method of claim 1, further comprising irradiating the item with UV-C radiation after the compartment has closed.

6. The method of claim 1, further comprising:
determining, by the server, one or more time slots during which the customer may be present at the bank of compartments without the staff member, another staff member or another customer being present at the bank of compartments; and
receiving, by the server, a selection from the customer of one of the time slots.

7. The method of claim 1, further comprising:
determining, by the server, one or more time slots during which the customer may be present at the bank of compartments so that there is at least a predetermined spacing between the compartment and another compartment that the staff member, another staff member or another customer is using; and
receiving, by the server, a selection from the customer of one of the time slots.

8. The method of claim 1, further comprising:
receiving, by the server, an indication that the staff member will process the item; and
opening, by instruction from the server, the compartment.

9. The method of claim 8, further comprising:
receiving, by the server, an indication that the staff member has rejected the return of the item;
determining, by the server, that the item has been replaced in the compartment;
closing, by instruction from the server, the compartment; and
informing, by the server, the customer to collect the item.

10. The method of claim 8, further comprising:
receiving, by the server, an indication that the staff member has accepted the return of the item;
again closing, by instruction from the server, the compartment; and
informing, by the server, the customer of acceptance of the return of the item.

11. The method of claim 10, further comprising switching on UV-C radiation inside the compartment after again closing the compartment.

12. The method of claim 1, further comprising:
detecting that the item comprises an unsafe material; and
alerting the staff member of the unsafe material.

13. A system for facilitating a return by a customer of an item that has been purchased, comprising:
a bank of compartments; and
a server comprising a processor and computer-readable memory storing computer-readable instructions which, when executed by the processor cause the server to:
establish a communication link with the customer;
determine that the customer is present at the bank of compartments;
open a compartment of the bank of compartments;
determine that the item has been placed in the compartment;
instruct the compartment to close;
inform a staff member that the item has been returned; and
identify the compartment to the staff member.

14. The system of claim 13, comprising a UV-C light source inside the compartment, wherein the server is configured to control the UV-C light source to sanitize:
an interior of the compartment when the item is not in the compartment; or
the item when it is in the compartment.

15. The system of claim 13, comprising:
a door that opens a rear of the compartment; and
another door that opens a front of the compartment.

16. A non-transitory, computer-readable medium storing computer-readable instructions, which when executed by a processer, cause a server to:
establish a communication link with the customer;
determine that the customer is present at a bank of compartments;
open a compartment of the bank of compartments;
determine that the item has been placed in the compartment;
instruct the compartment to close;
inform a staff member that the item has been returned; and
identify the compartment to the staff member.
